(19) United States
(12) Patent Application Publication
(10) Pub. No.: US 2011/0313504 A1
(43) Pub. Date: Dec. 22, 2011

(54) DEVICE AND METHOD TO PREVENT OR TREAT OUTFLOW VEIN STENOSIS OF AN ARTERIOVENOUS FISTULA CONSTRUCTED WITH A SYNTHETIC VASCULAR GRAFT

(76) Inventors: Arthur L. Golding, Los Angeles, CA (US); Daniel Allen Cota, San Diego, CA (US)

(21) Appl. No.: 13/150,684

(22) Filed: Jun. 1, 2011

Related U.S. Application Data
(60) Provisional application No. 61/350,621, filed on Jun. 2, 2010.

(51) Int. Cl. A61F 2/84; A61F 2/82

(52) U.S. Cl. 623/1.11; 623/1.35

(57) ABSTRACT

Systems and methods are provided that reduce or prevent the occurrence of NIH, VR and the resulting VS in the VOT of a GAVF. The system may protect the venous outflow tract of a GAVF from the deleterious physical and biological factors that induce: pathological cellular and biochemical responses which cause neointimal hyperplasia, venous remodeling, thrombosis, venous stenosis, and GAVF-vein anastomotic stenosis, including: a fenestrated expandable stent; and a tubular extension extending from the fenestration. The method protects the venous outflow tract of a GAVF from the deleterious physical and biological factors that induce: pathological cellular and biochemical responses which cause neointimal hyperplasia, venous remodeling, thrombosis, venous stenosis, and GAVF-vein anastomotic stenosis, and include steps of inserting the device into a vein to be used as a VOT; expanding the device; and joining the tubular extension of the device to a GAVF.

### CROSS-REFERENCE TO RELATED APPLICATIONS

This application claims benefit of priority to U.S. Provisional Patent Application Ser. No. 61/350,621, filed Jun. 2, 2010, entitled DEVICE AND METHOD TO PREVENT OR TREAT OUTFLOW VEIN STENOSIS OF AN ARTERIOVENOUS FISTULA CONSTRUCTED WITH A SYNTHETIC VASCULAR GRAFT, owned by the assignee of the present invention and herein incorporated by reference in its entirety.

### BACKGROUND

Constructing and maintaining satisfactory Vascular Access (VA) for Hemodialysis (HD) in patients with End Stage Renal Disease (ESRD) is a major problem. The Autogenous Arteriovenous Fistula (AAVF) and the Graft Arteriovenous Fistula (GAVF) are the best available techniques for VA at present. The AAVF is constructed by anastomosing (joining by means of sutures) a suitable vein to an appropriate artery. The GAVF is constructed by anastomosing a tubular graft of synthetic material of various diameters and lengths to a suitable vein and an appropriate artery which may be at considerable distance from each other. The GAVF to vein anastomosis and the segment of vein that receives the blood flow from the GAVF are designated the Venous Outflow Tract (VOT). A significant percent of ESRD patients do not have adequate veins for an AAVF and require a GAVF for chronic HD. The VOT of the GAVF becomes stenotic (narrowed) due to the formation of pathological tissue originating from the vein wall termed neointimal hyperplasia (NIH), the hypertrophy and thickening of the vein wall termed venous remodeling (VR), and the deposition of cellular elements, proteins and other biochemical substances from the blood stream. The resulting Venous Stenosis (VS) occurs within months following construction of the GAVF and may progress rapidly causing reduced blood flow thru the VOT and eventual thrombosis of the GAVF.

The GAVF must be monitored using flow, pressure and venous resistance measurements and imaging modalities (ultrasound, angiography) in order to evaluate the degree of VS and to intervene prior to thrombosis occurring. Attempts to reduce the degree of VS using interventional radiological procedures (angiography, angioplasty and stent placement) must be done frequently and repeatedly to maintain adequate blood flow rates and surgical procedures are required when the radiological procedures are not successful in correcting the problem. The progressive reduction of blood flow and resulting thrombosis of the GAVF presents a more difficult problem requiring the use of mechanical and/or biochemical interventions to remove the thrombotic material in addition to repairing the areas of VS. The damage to the GAVF from the repeated needle punctures required for HD further complicates the procedures to remove the thrombotic material.

The etiology of VS in GAVF has been studied in animal models and there are numerous clinical studies describing its etiology, progression and treatment. Unfortunately, the available treatments, both radiological and surgical, at best provide temporary improvement because of recurrent NIH and VR. These procedures must be repeated often and eventually fail requiring creation of a new GAVF. There are no methods available at present to prevent the occurrence of VS in the VOT of a GAVF for those ESRD patients using a GAVF for HD.

### SUMMARY OF THE INVENTION

In one aspect, the invention is directed towards a device to protect the venous outflow tract of a GAVF from the deleterious physical and biological factors that induce: pathological cellular and biochemical responses which cause neointimal hyperplasia, venous remodeling, thrombosis, venous stenosis, and GAVF-vein anastomotic stenosis, including: a fenestrated expandable stent; and a tubular extension extending from the fenestration.

Implementations of the invention may include one or more of the following. The stent may be expandable and may be lined, covered, or incorporated. The tubular extension may be substantially straight. A diameter of the tubular extension may be substantially equal to a diameter of a GAVF to which it will be joined. The tubular extension may have a flow diffuser segment. The flow diffuser segment may also have an enlarged diameter and fusiform shape at a proximal end that tapers towards a distal end to a cylinder having a diameter substantially equal to a diameter of a GAVF to which it will be joined. The lining, covering, or incorporating material of the stent may include a non-thrombogenic synthetic material. The stent may have a diameter between about 4 mm and 24 mm and a length between about 32 mm and 192 mm. The tubular extension may have a diameter between about 4 mm and 24 mm, and may extend from the stent a distance between about 4 cm and 10 cm. The tubular extension may extend from the stent at an angle between about 10 and 30 degrees, such as between about 10 and 20 degrees, such as about 15 degrees. The stent may be a lined stent having an internal surface including a synthetic material, where a thickness and porosity of the synthetic material is chosen so as to prevent physical and biochemical factors that can induce NIH and VR from affecting the vein wall. The stent may be a covered stent having an external surface made of a synthetic material, where a thickness and porosity of the synthetic material is chosen so as to prevent physical and biochemical factors that can induce NIH and VR from affecting the vein wall. The stent may be an incorporated stent having a synthetic material that partially or fully incorporates the stent within its substance, where a thickness and porosity of the synthetic material is chosen so as to prevent physical and biochemical factors that can induce NIH and VR from affecting the vein wall. The stent may include struts configured to enable the stent to be compressed for insertion and expanded for deployment. The device may further include a delivery system, the delivery system including a balloon, the balloon disposed in an uninflated configuration within the compressed stent, and the balloon configured to be inflated to deploy the stent. A proximal length of the stent, from a proximal extent of the fenestration, may be between about four and six times a diameter of the stent, and a distal length of the stent, from a distal extent of the fenestration, may be about 2 times the diameter of the stent.

In another aspect, the invention is directed to a method of protecting the venous outflow tract of a GAVF from the deleterious physical and biological factors that induce: pathological cellular and biochemical responses which cause neointimal hyperplasia, venous remodeling, thrombosis, venous stenosis, and GAVF-vein anastomotic stenosis, including: inserting the above device into a vein to be used as a VOT; expanding the device; and joining the tubular extension of the device to a GAVF.

### BRIEF DESCRIPTION OF THE DRAWINGS

FIG. 1 illustrates a top down view of a Lined/Covered/Incorporated Stent.

FIG. 2 illustrates a lateral view of a Lined/Covered/Incorporated Stent incorporating a Diffuser Tubular Extension.

FIG. 3 illustrates a lateral view of a Lined/Covered/Incorporated Stent incorporating a Straight Tubular Extension.

FIG. 4 illustrates another lateral view of a Lined/Covered/Incorporated Stent.

FIG. 5 illustrates another lateral view of a Lined/Covered/Incorporated Stent.

FIG. 6 illustrates a top down view of a Diffuser Tubular Extension.

FIG. 7 illustrates a top down view of a Straight Tubular Extension.

FIG. 8 illustrates a cross section oblique view of a device according to the principles described having a Diffuser Tubular Extension.

FIG. 9 illustrates a cross section oblique view of a device according to the principles described having a Straight Tubular Extension.

FIGS. 10(A) and (B) illustrate top down and lateral views of a suture ring and skirt.

FIG. 11 illustrates a perspective view of a suture ring and skirt.

### DETAILED DESCRIPTION

In the description below, various numerical values are given to provide examples of suitable parameters of the disclosed devices. It will be understood that these numerical values are purely exemplary, and that, in certain cases, other values may also be employed.

A device is provided that reduces or prevents the occurrence of NIH, VR and the resulting VS in the VOT of a GAVF. In order to better understand the rational for the device it is important to summarize the experimental and clinical studies that have defined the complex etiologic factors that produce VS of the VOT. These etiological factors include:

1) Physical factors caused by surgical instruments and manipulation including: dissection of perivenous tissue, traumatic compression of the vein wall, suturing of the GAVF to vein, and mechanical destruction of the vein's endothelial surface.
2) Physical factors inherent in the use of a GAVF including: compliance mismatch between the GAVF material and the vein wall, diameter mismatch between the GAVF and vein, and construction of an inappropriate anastomotic angle between the GAVF and vein resulting in deleterious blood flow patterns.
3) Nonphysiologic blood flow parameters within the VOT: high velocity, rapid acceleration, excessive intraluminal and transluminal pressures, abnormal blood flow patterns that result in turbulence, vein wall vibration, vortices, stagnation, fluid separation within the blood stream, abnormal shear stress at the blood-endothelial interface, and abnormal wall stress within the vein wall.

These multiple physical factors not only produce direct tissue damage but also induce, by mechanical transduction, the following pathological cellular and biochemical responses that cause NIH, VR and thrombus formation: endothelial dysfunction, cell migration and morphological alteration, release of multiple mediators of inflammation and oxidative stress, production of inappropriate growth factors, formation of excessive intercellular matrix, neovascularization of the vein wall and the developing NIH, deposition of cellular elements and proteins, and destruction of cells (endothelial and smooth muscle) and tissue components (elastin and collagen).

Unfortunately, the pathological cellular and biochemical responses noted above become more deleterious in the presence of the existing metabolic abnormalities caused by ESRD including: inadequate endothelial nitric oxide production, increased production of free radicals, inflammatory cytokines, and activated macrophages.

Present attempts to reduce or prevent the occurrence and extent of NIH and VR and reduce thrombus formation include experimental and clinical studies using several drugs, enzymes and gene modifiers that may alter the pathological cellular and biochemical responses that cause NIH and VR and thrombus formation.

Implementations of the present device may protect the vein wall from the multiple physical factors noted above that induce these pathological cellular and biochemical responses and may also provide a drug, enzyme or gene modifier component to reduce or prevent those biological responses that produce NIH and VR and thrombus formation should they occur. The device, in addition, may function as a stent of sufficient diameter and circumferentially outward directed radial force to prevent VS by its placement within the lumen of the VOT of the GAVF.

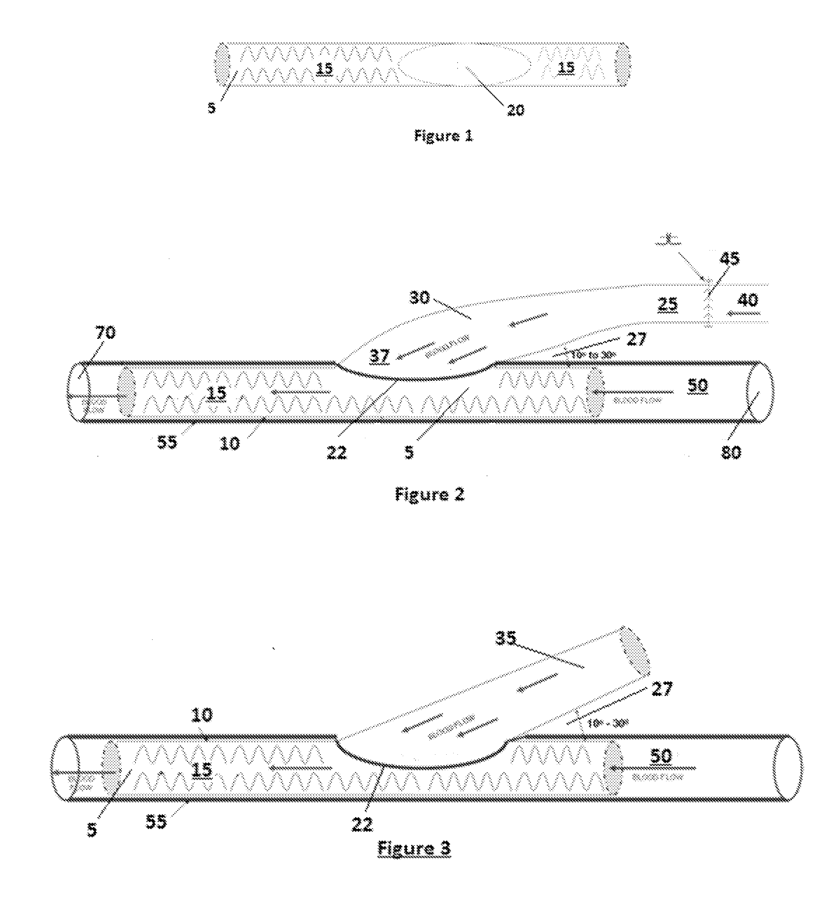

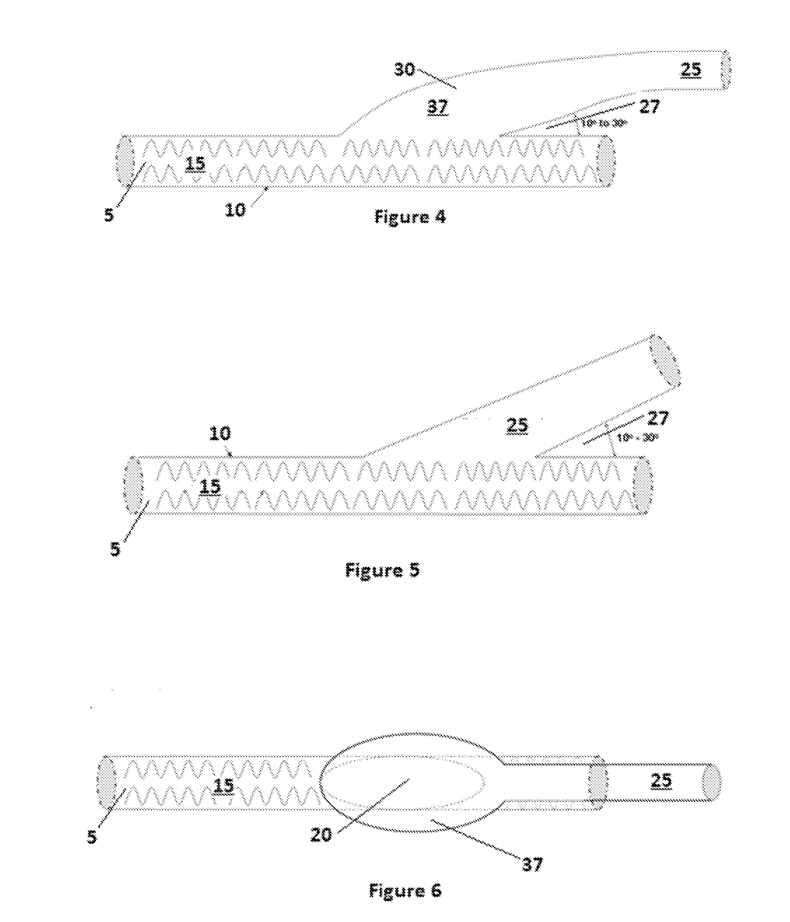

The implementations of the invention may provide a Cylindrical Fenestrated Expandable Lined, Covered or Incorporated Stent 5 (L/C/IS)(FIG. 1) with an integral unstented Tubular Extension 25 (TE) (FIG. 2) originating from the L/C/IS 5 at the site of the L/C/IS fenestration (opening) 20 (FIGS. 4, 5). The Stent may be composed of any of several metals or metal alloys configured in one of many available structural configurations. The lining, covering or incorporating material of the Stent may consist of a nonthrombogenic synthetic material. The TE orifice 20 and L/C/IS 5 fenestration are congruent and may be positioned approximately in the middle third of the overall length of the L/C/IS 5. The L/C/IS 5 may be available with several external diameters varying between about 4 mm. and 24 mm. and in several lengths varying between about 32 mm and 192 mm and the TE 25 may be available in multiple configurations and several external diameters varying between about 4 mm and 24 mm. The TE 25 in one implementation may have an enlarged diameter and fusiform shape 30 (FIG. 2) proximally and taper towards its distal end to a cylinder with a diameter that may be substantially equal to the diameter of the GAVF 40 to which it will be joined (FIG. 2, 4). The TE 25 in another implementation may be of uniform diameter and not taper toward its distal end but consist of a cylindrical tube of uniform diameter throughout its length that may be substantially equal in diameter to the diameter of the GAVF 40 to which it will be joined (FIG. 3, 5). The TE 25 may extend from the L/C/IS 5 a length varying between about 4 cm and 10 cm at a preferred angle 27 of 15 degrees from the L/C/IS 5. Various other angulations 27 may be available varying between about 10 degrees and 30 degrees and may be selected in each instance of use depending on the placement of the GAVF 40 to which it will be joined.

In more detail, the L/CIS 5 may be constructed of a thin-walled synthetic material that forms a cylinder 10, circular in cross section throughout its length, composed of any number of synthetic materials which can provide a nonthrombogenic surface in one of three configurations in relation to the Stent: as an internal surface of the device forming a Lined Stent (LS); as an external surface of the device forming a Covered Stent (CS); or the synthetic material may partially or completely incorporate the Stent within its substance forming an Incorporated Stent (IS). Thus the Stent struts 15 may be exposed on the external surface of the LS 5, or the Stent struts 15 may be exposed within the lumen of the CS or the Stent struts 15 may be fully or partially incorporated within the synthetic material forming the wall 10 of the device. The synthetic material may be of appropriate thickness and porosity to prevent the physical and biochemical factors that can induce NIH and VR from exerting their effects on the vein wall 55.

The Stent 5 may include any number of various diameter struts 15 that may be composed of various metals or metallic alloys arranged in any number of configurations (FIGS. 1, 4, 5). The struts 15 are of such design so as to enable the Stent 5 to be compressed and temporarily reduced in diameter to allow ease of placement within the lumen of a vein 50 and subsequently to be expandable to a variable diameter within the vein 50. This expansion may compress either the outer surface of the Stent struts 15 against the inner surface of the vein wall 55 when using a Lined Stent (LS) 5 or the outer surface of the synthetic material when using a Covered Stent (CS) 5 or the combined stent struts 15 and synthetic material when using an Incorporated Stent (IS) (FIGS. 2,3). The expansion may be accomplished by means of several available methods such as an inflatable “balloon” temporarily positioned within the lumen of the L/C/IS or by the use of a Stent composed of a self-expanding metallic alloy material. The struts 15 may or may not be coated or otherwise bonded to various materials containing various chemicals including drugs, enzymes and gene modifiers singly or in combination whose presence and/or by their elution may prevent the vein from undergoing those responses that may produce NIH and VR and result in VS and/or prevent the deposition of platelets, fibrin or other blood components that may induce thrombus formation. The proximal length of the Stent 5, extending from the proximal 70 extent of the fenestration, may be approximately four (4) to six (6) times the diameter of the stent (4 mm to 24 mm in diameter) and thus may vary between about 16 mm and 144 mm in length and the distal 80 length of the Stent 5 extending from the distal 80 extent of the fenestration 20 may be about two (2) times the diameter of the Stent 5 and thus may vary between about 8 mm and 48 mm in length.

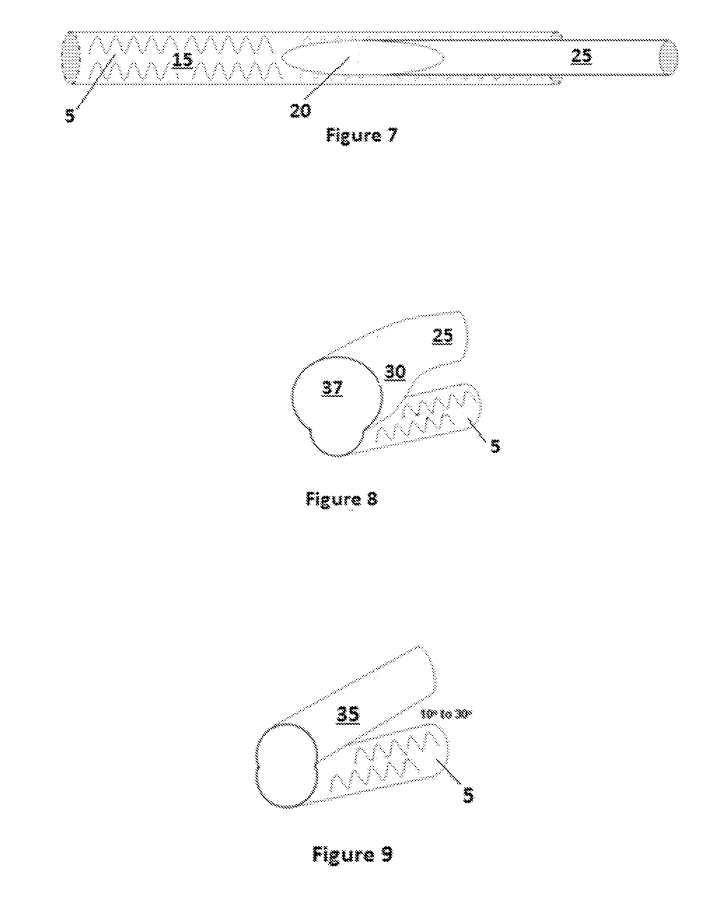

The TE 25 may be composed of the same synthetic material as that of the L/C/IS wall 10 and may be formed as an integral extension of that wall 10 (FIGS. 4, 5). The orifice 20 of the TE 25 at its junction with the LS 5 fenestration may substantially equal the dimensions of the fenestration in the wall 10 of the LS 5. The TE orifice/L/C/IS fenestration 20 may be elliptical or circular in shape (FIGS. 1, 7) and may vary between about 1.0 cm and 3.0 cm in length and may have a width substantially equal to the diameter of the L/C/IS 5 of which it is an integral part (FIG. 1). The dimensions of the orifice/fenestration 20 may vary in proportion to the diameters of the TE 25 and L/C/IS 5.

The TE 25 may include in one implementation an expanded flow diffusing configuration 30 at and adjacent to its junction with the L/C/IS 5. The flow diffusing segment 37 may be either semicircular or elliptical in cross section and tapering distally so that it becomes circular in cross section and may be substantially equal in diameter to the diameter of the GAVF 40 to which it may be joined. The length, height and width of the flow diffuser segment 37 of the TE 25 may vary with the dimensions of the L/C/IS 5, the TE 25, and the orifice/fenestration 20, and may be of various configurations including but not limited to fusiform, hooded, bulbous and conical shapes (FIGS. 2, 4, 6, 8). The purpose of the flow diffuser segment 37 is to favorably alter the blood flow velocity, acceleration, intraluminal pressure, shear stress, wall stress and blood flow patterns. These favorable alterations may prevent the previously described physical factors from affecting the more proximal 70 and distal 80 segments of the unstented vein of the VOT beyond the extent of the L/C/IS 5 (FIGS. 2, 3) and thereby may reduce or prevent VS in those segments. In addition it may reduce the deposition of blood, cellular and protein elements, and reduce the potential for thrombus formation within the L/C/IS 5 and TE 25.

The TE 25, in another implementation, may consist of a cylinder of uniform diameter throughout its length 35, thereby lacking a flow diffuser segment (FIG. 3, 5, 7, 9). The TE 25 may be composed of the same synthetic material as that of the LS wall 10 and may be formed as an integral extension of that wall. The orifice 20 of the TE 25 at its junction with the L/C/IS 5 fenestration may substantially equal the dimensions of the fenestration in the wall 10 of the L/C/IS. The TE orifice/L/C/IS fenestration 20 may be elliptical or circular in shape and may vary between about 1.0 cm and 3.0 cm in length and have a width, e.g., substantially equal to the diameter of the L/C/IS 5 of which it is an integral part. The dimensions of the orifice/fenestration 20 may vary in proportion to the diameters of the TE 25 and L/C/IS 5. The TE 25 may be circular in cross section and may be substantially equal in diameter to the diameter of the GAVF 40 to which it may be joined.

The TE 25 in all implementations will be joined to the blood outflow (venous) segment of a GAVF 40 to provide the means for transporting the blood flow from the GAVF 40 to the VOT of the patient (FIG. 2). The joining may be done after insertion of the L/C/IS 5 within the vein 50 that will function as the VOT. The TE 25 and GAVF 40 may be of substantially equal diameters and the joining or anastomosis may be done by available mechanical or “hand” suture techniques in order to construct a precise and smooth junction 45. The TE 25 may be joined to the GAVF 40 at varying distances from the TE's origin depending on the length of TE 25 selected and the placement of the GAVF 40 in each instance of use.

In one exemplary method of use of an implemented device, the device may be inserted into the vein 50 to be used as the VOT using a longitudinal incision. The incision used for insertion may be of sufficient length for ease of placement of the L/C/IS 5 and TE 25. The L/C/IS 5 may be expanded after placement in the VOT and before joining the TE 25 to the GAVF 40. The expanded L/C/IS 5 may fill the vein lumen completely, abut the inner surface of the vein wall 55 and extend both proximally and distally within the vein lumen from the incision site. The TE 25 as noted may be available in various angulations 27 and may therefore exit the vein 50 thru the incision at an angle 27 that may vary between about 10 degrees and 30 degrees to the L/C/IS 5 and vein 50 (FIGS. 2, 3). The angulation selected being dependent on the placement of the GAVF 40 to which it will be joined as previously described. Utilizing an angulation between about 10 and 30 degrees may provide the most physiological hemodynamic parameters for blood flow within the VOT. Leakage of blood may not occur from the incision site in the vein due to the expansion of the synthetic material and Stent struts 15 forming the wall 10 of the L/C/IS 5 and the presence of the TE 25 exiting thru the incision in the vein 22 both acting thereby to isolate the blood flow within the TE 25 and L/CS 5 from the incision. The L/C/IS 5 and TE 25 may adhere to the edges of the incision 22 and abut against the vein wall 55 and may occlude the incision by their presence and position (FIGS. 2, 3).

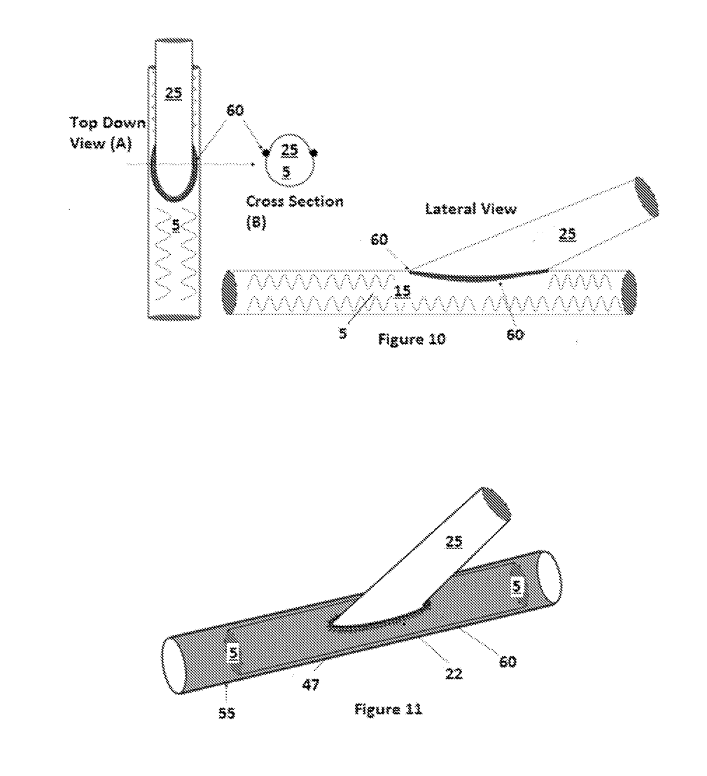

In those instances when there may be concern that leakage of blood from the incision in the vein 22 may occur due to the wall of the vein being fibrotic, rigid or otherwise damaged, thereby preventing coaptation of the edges of the vein incision and/or the inner surface of the vein wall 55 to the external surface of the L/C/IS 5 and TE 25, another implementation of the device may be utilized. In this implementation a sewing ring or sewing skirt 60 may be present along the entire perimeter of the external surface of the junction of the L/C/IS 5 and TE 25 (FIG. 10, 11). The ring or skirt 60 thus formed may be composed of the same synthetic material comprising the L/C/IS 5 and TE 25 and may be an integral part of and extend outward from the external surface of the device. The ring configuration may extend a distance of 2 mm to 4 mm from that external surface and may have a thickness of 2 mm (FIG. 10). The skirt configuration may extend a distance of 3 mm. to 4 mm. from that external surface and may have a thickness of 2 mm. (FIG. 10). The ring or skirt 60 may be used to enable the device to be sutured to the edge of the incision in the vein 22 by placing a continuous or an interrupted row of sutures 47 between the ring or skirt 60 and the vein wall 55 (FIG. 10). This may provide a hemostatic closure of the incision in the vein wall 55 thru which the device is initially inserted and may not alter the function of the device.

Certain implementations of the invention may include several aspects that provide significant functionality. The L/C/IS 5 may protect the vein wall 55 from the direct effects of the physical factors that induce the pathological cellular and biochemical responses which result in NIH and VR. The L/C/IS 5 may eliminate the GAVF to vein anastomosis and thereby significantly reduce surgical trauma to the vein 50 and the TE 25 may eliminate the GAVF to vein compliance and diameter mismatch. In the described implementations the TE configuration and the angle 27 at which the TE 25 joins the L/C/IS 5 may favorably alter the blood flow patterns, intraluminal pressure, flow rates, and shear and wall stress within the proximal and distal unstented segments of the VOT beyond the extent of the L/C/IS 5. The drugs, enzymes and gene modifiers which may be present on the struts 15 of the L/C/IS 5 may act to reduce or prevent the pathological cellular and biochemical responses that produce NIH and VR and thrombus formation. In addition, the L/C/IS 5, when expanded within the vein lumen, may provide a circumferentially outward directed radial force that may prevent luminal narrowing and VS.

Implementations of the device may be particularly useful when the GAVF 40 is initially constructed and the selected vein 50 and the VOT has not yet been affected by the previously noted factors that induce NIH and VR. The use of the device to protect the undamaged VOT may significantly enhance its capability to reduce or prevent the development of VS within the VOT.

The device may also be used when VS of the VOT is present due to a previously constructed GAVF. In this method of use the proximal segment 70 of the L/C/IS 5 may extend beyond the stenotic and/or thrombosed segment of the VOT to an area of undamaged vein 50 and the entire length of the L/C/IS 5 must be expanded to its full diameter which must be equivalent to the diameter of the proximal undamaged vein 50. The same extent of expansion must be accomplished in that portion of the L/C/IS 5 positioned within the stenotic segment of the VOT.

While the system and method have described several specific implementations in which the invention may be practiced, numerous other variations are possible. Accordingly, the present invention is not limited to only those implementations described above.

1. A device to protect the venous outflow tract of a GAVF from the deleterious physical and biological factors that induce: pathological cellular and biochemical responses which cause neointimal hyperplasia, venous remodeling, thrombosis, venous stenosis, and GAVF-vein anastomotic stenosis, comprising:
a fenestrated expandable stent; and
a tubular extension extending from the fenestration.

2. The device of claim 1, wherein the stent is expandable and is lined, covered, or incorporated.

3. The device of claim 1, wherein the tubular extension is substantially straight.

4. The device of claim 3, wherein a diameter of the tubular extension is substantially equal to a diameter of a GAVF to which it will be joined.

5. The device of claim 1, wherein the tubular extension has a flow diffuser segment.

6. The device of claim 5, wherein the flow diffuser segment has an enlarged diameter and fusiform shape at a proximal end that tapers towards a distal end to a cylinder having a diameter substantially equal to a diameter of a GAVF to which it will be joined.

7. The device of claim 2, wherein the lining, covering, or incorporating material of the stent includes a non-thrombogenic synthetic material.

8. The device of claim 1, wherein the stent has a diameter between about 4 mm and 24 mm and a length between about 32 mm and 192 mm.

9. The device of claim 1, wherein the tubular extension has a diameter between about 4 mm and 24 mm.

10. The device of claim 1, wherein the tubular extension extends from the stent a distance between about 4 cm and 10 cm.

11. The device of claim 1, wherein the tubular extension extends from the stent at an angle between about 10 and 30 degrees.

12. The device of claim 11, wherein the tubular extension extends from the stent at an angle between about 10 and 20 degrees.

13. The device of claim 12, wherein the tubular extension extends from the stent at an angle of about 15 degrees.

14. The device of claim 2, wherein the stent is a lined stent having an internal surface comprised of a synthetic material, wherein a thickness and porosity of the synthetic material is chosen so as to prevent physical and biochemical factors that can induce NIH and VR from affecting the vein wall.

15. The device of claim 2, wherein the stent is a covered stent having an external surface comprised of a synthetic material, wherein a thickness and porosity of the synthetic material is chosen so as to prevent physical and biochemical factors that can induce NIH and VR from affecting the vein wall.

16. The device of claim 2, wherein the stent is an incorporated stent having a synthetic material that partially or fully incorporates the stent within its substance, wherein a thickness and porosity of the synthetic material is chosen so as to prevent physical and biochemical factors that can induce NIH and VR from affecting the vein wall.

17. The device of claim 2, wherein the stent comprises struts configured to enable the stent to be compressed for insertion and expanded for deployment.

18. The device of claim 17, further comprising a delivery system, the delivery system including a balloon, the balloon disposed in an uninflated configuration within the compressed stent, and the balloon configured to be inflated to deploy the stent.

19. The device of claim 1, wherein a proximal length of the stent, from a proximal extent of the fenestration, is between about four and six times a diameter of the stent, and wherein a distal length of the stent, from a distal extent of the fenestration, is about 2 times the diameter of the stent.

20. A method of protecting the venous outflow tract of a GAVF from the deleterious physical and biological factors that induce: pathological cellular and biochemical responses which cause neointimal hyperplasia, venous remodeling, thrombosis, venous stenosis, and GAVF-vein anastomotic stenosis, comprising:
inserting a device according to claim 1 into a vein to be used as a VOT;
expanding the device; and
joining the tubular extension of the device to a GAVF.
